(19) United States
(12) Patent Application Publication
(10) Pub. No.: US 2006/0135807 A1
(43) Pub. Date: Jun. 22, 2006

(54) Process for preparing esters of 3-amino-4-halobenzoic acid

(76) Inventors: Thomas Elbert Shanks, Piney Flats, TN (US); Robert Joseph Maleski, Kingsport, TN (US)

Correspondence Address: Michael J. Blake;Eastman Chemical Company, P.O. Box 511, Kingsport, TN, 37662-5075, US

(21) Appl. No.: 11/024,913

(22) Filed: Dec. 20, 2004

(51) Int. Cl. C07C 229/54

(52) U.S. Cl. 560/19

(57) ABSTRACT

Disclosed is a process for producing good yields of high quality esters of 3-amino-4-halobenzoic acid by contacting the subject acid with an alkyl halide or sulfonate in a solvent in the presence of an aqueous alkali metal hydroxide and a phase transfer catalyst comprising tetrasubstituted ammonium or phosphonium salt.

### FIELD OF THE INVENTION

The present invention relates to an improved process for preparing esters of 3-amino-4-halobenzoic acid in good yields with high quality.

### BACKGROUND OF THE INVENTION

Certain esters of 3-amino-4-chlorobenzoic acid are important intermediates in the synthesis of photographic couplers. See U.S. Pat. No. 3,752,072. A process for making the intended esters may be found in U.S. Pat. No. 4,135,050. Therein, a short chain ester (e.g., a methyl ester) of an anthranilic acid is subjected to transesterification with an alcohol. The process in the '050 patent uses potassium carbonate as the transesterification catalyst.

An additional process for making such esters directly from 3-amino-4-chlorobenzoic acid has been disclosed in U.S. Pat. No. 5,908,955. In the '955 patent, the benzoic acid is reacted with the corresponding alkyl halide in the presence of a basic carbonate. The process disclosed in the '955 patent requires heating the benzoic acid and a solvent and then adding a basic carbonate and finally adding, after further heating, an alkyl halide. The '955 patent, which stresses the order of addition for the reactants, recites that the solvent for use therein is a polar non-protic organic solvent such as N,N-dimethylformamide, N, N-dimethylacetamide, or dimethylsulfoxide. Although the '955 patent reports high yields of good quality products, the required solvents are expensive, toxic, and difficult to recover for re-use. Notably, both the '050 and the '955 patents employ inorganic, basic carbonates that are quite expensive as compared to other basic materials, such as alkali metal hydroxides.

Thus, there exists a need for a process that produces good quality materials at good yields but that uses inexpensive, non-toxic, and easily recovered solvents and inexpensive bases.

### BRIEF SUMMARY OF THE INVENTION

We have found that good yields of high quality esters of 3-amino-4-halobenzoic acid can be obtained by contacting the subject acid with an alkyl halide or sulfonate in the presence of an alkali metal hydroxide under phase transfer conditions:

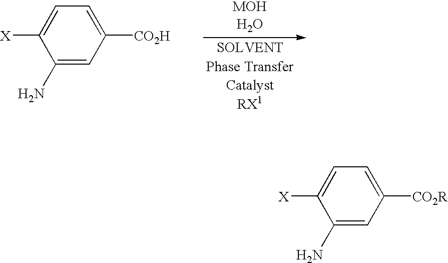

In the above equation, M is an alkali metal cation such as potassium, sodium or lithium. The solvent can be any water-immiscible solvent in which the product is soluble and that does not degrade under the reaction conditions. The phase transfer catalyst can be any of the commonly available tetrasubstituted ammonium or phosphonium salts. The alkylating agent is represented by RX¹, in which X¹ can be any good halide or sulfonate leaving group such as chloride, bromide, iodide, methanesulfonate, p-toluenesulfonate, or benzene sulfonate. X above is a halogen.

The corresponding alkali metal salt of the 3-amino-4-halobenzoic acid starting material may be used in the above reaction, but under those circumstances the need for the hydroxide base can be considerably reduced or even eliminated.

### DETAILED DESCRIPTION OF THE INVENTION

As stated above, the present invention is a process for producing good yields of high quality esters of 3-amino-4-halobenzoic acid by contacting the subject acid with an alkyl halide or alkyl sulfonate in a solvent in the presence of an alkali metal hydroxide under phase transfer conditions. Of particular interest are the esters of 3-amino-4-chlorobenzoic acid. The term “phase transfer conditions,” which will be understood by those of skill in the art, is meant to connote the wide variety of conditions that employ phase transfer catalysts, such as is described in, e.g., Starks, Liotta, and Halpern, “Phase Transfer Catalysis”, Chapman and Hall, 1997. Suitable benzoic acid starting materials are widely commercially available; the corresponding alkali metal salt can be made by methods well known to those skilled in the art.

The alkyl halide or alkyl sulfonate for use herein may be represented by the formula RX¹. The R group can be any branched or straight chain C₆-C₂₀ alkyl group and may be unsubstituted or substituted with one or more C₅-C₈ aryl or C₅-C₈ cycloalkyl groups, or substituted with any moiety that is itself not labile under the reaction conditions. Typical alkyl groups represented by R include hexyl, octyl, 2-ethylhexyl, dodecyl, or hexadecyl. As noted above, X¹ can be any good leaving group, which includes a halide such as chloride, bromide, or iodide, or a sulfonate such as methanesulfonate, p-toluenesulfonate, or benzene sulfonate. The amount of RX¹used should be at least enough to react with the benzoic acid salt added or generated in situ. Thus, typically about 1 to about 1.5 equivalents of RX¹ will be used relative to the benzoic acid or benzoic acid salt starting material, with about 1 to about 1.2 equivalents being preferred.

The skilled artisan will understand that each of the references herein to groups or moieties having a stated range of carbon atoms, such as “C₆-C₂₀ alkyl,” includes not only the C₆ group (hexyl) and C₂₀ group (icosanyl) end points, but also each of the corresponding individual groups within the range, such as each of the C₇, C₈, C₉ and C₁₀ groups, and so forth. In addition, it will be understood that each of the individual points within a stated range of carbon atoms may be further combined to describe sub-ranges that are inherently within the stated overall range. For example, the term “C₆-C₂₀-alkyl” includes not only the individual moieties C₆ through C₂₀, but also contemplates sub-ranges such as “C₈-C₁₆-alkyl.”

The alkali metal hydroxide for use herein may be either potassium hydroxide, sodium hydroxide or lithium hydroxide. The amount of alkali metal hydroxide base used in the reaction should be sufficient to neutralize the benzoic acid starting material. Typically, however, about 1 to about 1.5 equivalents relative to the benzoic acid are used, with about 1 to about 1.2 being preferred. If the alkali metal salt of the benzoic acid is used as the starting material, the base can be reduced to about 0.5 equivalents or less, with about 0.2 equivalents or less being preferred. Alternatively, the base can be substantially eliminated, in which case water alone is added to the reaction medium.

The amount of water present in the reaction medium can be any convenient amount that is sufficient to dissolve the alkali metal hydroxide. Typically, the amount of water present will be from about 0.75 to about 20 parts water per part of hydroxide base; preferably, water will be present from about 1 to about 10 parts per part of hydroxide base. When an alkali metal salt of a benzoic acid is used as a starting material, the amount of water used may be reduced to the amount necessary to dissolve the alkali metal salt.

The solvent for use in the present invention may be chosen from among any number of water-immiscible solvents that are of a sufficiently high boiling point to promote the reaction. Typical solvents include heptane or octane, or other high boiling alkanes; toluene or xylene, or other high boiling aromatic hydrocarbons; or any of a variety of water immiscible ketones such as methyl isobutyl ketone or methyl isoamyl ketone. Preferred solvents include toluene, xylene, or methyl isobutyl ketone; such solvents are readily available and inexpensive

The phase transfer catalyst used herein may be any of a number of commercially available tetrasubstituted ammonium or phosphonium salts. Examples include tetrabutylammonium bromide, benzyltriethylammonium bromide, tetrahexylammonium bromide, triocytlmethylammonium chloride, tetrabutylphosphonium bromide, and hexadecyltributylphosphonium bromide. Preferred catalysts for use herein include tetrabutylammonium bromide and tetrabutylphosphonium bromide. The amount of phase transfer catalyst is chosen such that the reaction can be completed in a reasonable amount of time. Typically about 2 to about 25 mole percent of catalyst is used based on the amount of benzoic acid starting material, with about 1 to about 15 mole percent preferred.

The reaction may be carried out by mixing all of the components with good agitation to create a reaction mixture and adjusting the temperature to the appropriate point. While the reaction does proceed by refluxing the 2-phase mixture, the boiling point of this mixture is fixed at the boiling point of the azeotropic mixture of the organic solvent and the water. In practice, it is best to remove the water via azeotropic distillation during the course of the reaction in order to increase and maintain the reaction temperature and thus cause the reaction to proceed faster. Under these circumstances the typical reaction temperatures are about 100° C. to about 180° C., with about 110° C. to about 140° C. being preferred. The reaction time for alkylating the benzoic acid or salt is dictated by the properties of the solvent, or the solvent-water mixture, but reaction conditions should, in general, be chosen such that the reaction is complete in about 1 to about 10 hours (hr), with about 2 to about 6 hrs being preferred. The order of addition of the reactants is not important.

The resulting organic phase will contain the intended product ester along with minor amounts of impurities such as the product in which the alkylation takes place on the nitrogen instead of the carboxylic acid group. The product ester may be isolated and purified by methods well-known to those skilled in the art, such as crystallization from a suitable solvent, such as heptane or octane.

The invention can be further illustrated by the following examples, although it will be understood that the examples are included for illustration purposes and are not intended to limit the scope of the invention.

### EXAMPLES

### Example 1

n-Dodecyl methanesulfonate: 186.3 g of 1-dodecanol, 500 g of acetone, and 106.1 g of triethylamine was treated at 20-30° C. with 120.2 g of methanesulfonyl chloride. The slurry was warmed to 50-55° C. and held at that temperature for 1 hr. It was then cooled to room temperature and treated with 700 mL of water. Upon cooling to 0-5° C. the product crystallized and was collected by filtration and washed on the filter with 700 mL of cold water. After drying in a 40° C. vacuum oven the product weighed 252.4 g (96% weight yield) and assayed 90% by nmr. The assay yield was 86%.

### Example 2

n-Dodecyl-3-amino-4-chlorobenzoate: A mixture of 63 g of water, 12 g (0.15 moles) of 50% sodium hydroxide solution, 25.5 g (0.125 moles) of 3-amino-4-chlorobenzoic acid, 50 g of xylene, 43 g (100% basis, 0.163 moles) of n-dodecyl methanesulfonate and 2.2 g of tetrabutylphosphonium bromide (5 mole percent) were heated to reflux and water was removed by means of a Dean Stark trap. After 5 hr at reflux (140° C.) the reaction was judged complete by tic. The reaction was cooled to 80° C. and washed with water to remove salts. The xylene was removed on a roto-vap and the residue was crystallized from heptane to give 42.5 g (100% yield) of a solid whose nmr weight percent assay was 97.6%.

### Example 3

n-Hexadecyl-3-amino-4-chlorobenzoate: A mixture of 32.7 g (0.264 moles) 45% potassium hydroxide solution, 43 g (0.25moles) of 3-amino-4-chlorobenzoic acid, 200 g of methyl isobutyl ketone, 80.6g (0.264 moles) of 1-bromohexadecane and 4.4 g of tetrabutylphosphonium bromide (5 mole percent) were heated to reflux and water was removed by means of a Dean Stark trap. After 5 hr at reflux (115-120° C.) the reaction was judged complete by tic. The reaction was cooled to 90° C. and washed with water to remove salts. The solvent was removed on a roto-vap and the residue was crystallized from heptane to give 93.4 g (95% yield) of a solid whose nmr weight percent assay was 99.6%.

### Example 4

n-Dodecyl-3-amino-4-chlorobenzoate: A mixture of 16.4 g (0.125 moles) of 45% potassium hydroxide solution, 21.5 g (0.125 moles) of 3-amino-4-chlorobenzoic acid, 100 g of xylene, 32.8 g (0.132 moles) of 1-bromododecane, and 4.5 g of tetrabutylammonium bromide (12 mole percent) were heated to reflux and water was removed by means of a Dean Stark trap. After 5 hr at reflux (140° C.) the reaction was judged complete by tic. The reaction was cooled to 80° C. and washed with water to remove salts. The xylene was removed on a roto-vap. The residue weighed 43 g (100% yield) and was judged pure by tic and by nmr.

### Example 5

n-Hexadecyl-3-amino-4-chlorobenzoate: A mixture of 16.3 g (0.132 moles) 45% potassium hydroxide solution, 21.5 g (0.125 moles) of 3-amino-4-chlorobenzoic acid, 100 g of xylene, 40/3 g (0.132 moles) of 1-bromohexadecane and 4.5 g of benzyltriethylammonium bromide (10 mole percent) were heated to reflux and water was removed by means of a Dean Stark trap. After 3 hr at reflux (115-120° C.) the reaction was judged complete by tic. The reaction was cooled to 90° C. and washed with water to remove salts. The solvent was removed on a roto-vap and the residue was crystallized from heptane to give 40.7 g (83% yield) of a solid judged to be pure by nmr and by tic.

The invention has been described in detail with particular reference to preferred embodiments thereof, but it will be understood that variations and modifications can be effected within the spirit and scope of the invention.

1. A process for producing a 3-amino-4-halobenzoic acid ester which comprises contacting 3-amino-4-halobenzoic acid with an alkyl halide or sulfonate in the presence of an aqueous alkali metal hydroxide, a solvent, and a tetrasubstituted ammonium or phosphonium salt.

2. A process according to claim 1 wherein the acid is 3-amino-4-chlorobenzoic acid; the hydroxide is sodium hydroxide or potassium hydroxide; and the alkyl halide or sulfonate is represented by RX¹, wherein R is a branched or straight chain C₆-C₂₀ alkyl group, which may be unsubstituted or substituted with one or more C₅-C₈ aryl or C₅-C₈ cycloalkyl groups, and X¹ is a halide or a sulfonate.

3. A process according to claim 2 wherein R is a branched or straight chain C₆-C₂₀ alkyl group and X¹ is chloride, bromide, iodide, methanesulfonate, p-toluenesulfonate, or benzene sulfonate.

4. A process according to claim 3 wherein R is hexyl, octyl, 2-ethylhexyl, dodecyl or hexadecyl.

5. A process according to claim 3 wherein the solvent is a high boiling alkane, a high boiling aromatic hydrocarbon, a water immiscible ketone or a mixture thereof.

6. A process according to claim 5 wherein the ammonium or phosphonium salt is tetrabutylammonium bromide, benzyltriethylammonium bromide, tetrahexylammonium bromide, triocytlmethylammonium chloride, tetrabutylphosphonium bromide and the solvent is heptane, octane, toluene, xylene, methyl isobutyl ketone, methyl isoamyl ketone or a mixture thereof.

7. A process according to claim 6 wherein the ammonium or phosphonium salt is tetrabutylammonium bromide or tetrabutylphosphonium bromide.

8. A process according to claim 2 which further comprises the step of isolating the ester by crystallizing the ester in heptane or octane.

9. A process for producing a 3-amino-4-chlorobenzoic acid ester which comprises contacting 3-amino-4-chlorobenzoic acid with RX¹ in the presence of sodium hydroxide or potassium hydroxide, water, a solvent and a phase transfer catalyst, wherein R is a branched or straight chain C₆-C₂₀ alkyl group and X¹ is chloride, bromide, iodide, methanesulfonate, p-toluenesulfonate, or benzene sulfonate.

10. A process according to claim 9 wherein the R is hexyl, octyl, 2-ethylhexyl, dodecyl or hexadecyl; the catalyst is tetrabutylammonium bromide or tetrabutylphosphonium bromide; and the solvent is heptane, octane, toluene, xylene, methyl isobutyl ketone, methyl isoamyl ketone or a mixture thereof.

11. A process according to claim 10 which further comprises the step of distilling to remove water.

12. A process according to claim 11 which further comprises the step of isolating the ester by crystallizing the ester in heptane or octane.

13. A process for producing a 3-amino-4-chlorobenzoate ester which comprises contacting an alkali metal salt of 3-amino-4-chlorobenzoic acid with RX¹ in the presence of water, a solvent, and a tetrasubstituted ammonium or phosphonium salt, wherein R is a branched or straight chain C₆-C₂₀ alkyl group and X¹ is chloride, bromide, iodide, methanesulfonate, p-toluenesulfonate, or benzene sulfonate.

14. A process according to claim 13 wherein R is hexyl, octyl, 2-ethylhexyl, dodecyl or hexadecyl; the alkali metal salt of 3-amino-4-chlorobenzoic acid is a sodium or potassium salt; the ammonium or phosphonium salt is tetrabutylammonium bromide, benzyltriethylammonium bromide, tetrahexylammonium bromide, triocytlmethylammonium chloride or tetrabutylphosphonium bromide; and the solvent is heptane, octane, toluene, xylene, methyl isobutyl ketone, methyl isoamyl ketone or a mixture thereof.

15. A process for producing a 3-amino-4-chlorobenzoic acid ester which comprises:
contacting 3-amino-4-chlorobenzoic acid with RX¹ in the presence of water, sodium or potassium hydroxide, a solvent, and a phase transfer catalyst; and
distilling to remove water, wherein R is hexyl, octyl, 2-ethylhexyl, dodecyl or hexadecyl; X¹ is chloride, bromide, iodide, methanesulfonate, p-toluenesulfonate, or benzene sulfonate; the solvent is heptane, octane, toluene, xylene, methyl isobutyl ketone, methyl isoamyl ketone or a mixture thereof; and the catalyst is tetrabutylammonium bromide or tetrabutylphosphonium bromide.

16. A process according to claim 15 which further comprises the step of isolating the ester by crystallizing the ester in heptane or octane.
